(19) United States
(12) Patent Application Publication
(10) Pub. No.: US 2021/0213446 A1
(43) Pub. Date: Jul. 15, 2021

(54) SAMPLE ANALYSIS DEVICE

(71) Applicant: Novoheart Limited, Kowloon (HK)

(72) Inventors: Eugene K. Lee, Irvine, CA (US); Kevin D. Costa, Irvine, CA (US); Erin G. Roberts, Irvine, CA (US)

(21) Appl. No.: 17/144,083

(22) Filed: Jan. 7, 2021

Related U.S. Application Data
(60) Provisional application No. 62/958,838, filed on Jan. 9, 2020.

(51) Int. Cl. B01L 3/00; C12M 1/34

(52) U.S. Cl. CPC B01L 3/502707; B01L 2200/0652; C12M 41/36; B01L 3/502715

(57) ABSTRACT

The present disclosure relates to a sample analysis device for increasing measurement resolution of forces generated by a sample and method of use thereof. The sample analysis device comprises a body stretchable along a central longitudinal axis. The body includes at least one attachment region engageable with a sample and a plurality of stretchable portions spaced apart by joining regions. The body also includes at least one detectable datum for determining displacement. Each stretchable portion extends generally longitudinally and includes an offset region inclined to the longitudinal axis. The stretchable portions increase the compliance of the body within a predetermined measurement range.

### PRIORITY CLAIM

This application claims priority to U.S. Patent Application No. 62/958,838, filed on Jan. 9, 2020, the entire disclosure of which is incorporated by reference herein.

### FIELD

The present disclosure relates to a sample analysis device for increasing resolution of measurement of forces generated by a sample, particularly a biological sample from biological sources.

### BACKGROUND

The current preclinical development and screening of novel therapeutic agents along with understanding disease pathogenesis and associated therapies typically utilize animal models. However, due to their inherent physiological differences, animal models have proven to be less than ideal, especially in the prediction of drug induced toxicity in human cardiac muscle tissues. As a result of the inherent physiological differences, researchers have focused significant efforts on in vitro assays that employ human-based cell sources in an attempt to address some of the above disadvantages. However, this can be relatively complex in practice.

For example, in assays which model the human muscular system, engineered tissue constructs in bioreactors/platforms need to simulate the physiological environment native to the specific muscle tissue, including the complex three dimensional environment. There is also the added complication that measurement of the tissue construct needs to allow for desirable anisotropic properties (different physical properties according to the direction of measurement in an object).

Accordingly, although such tissue constructs have demonstrated responses similar to in vivo human physiology, the majority of these assays and their associated methodologies for quantifying muscle properties have been developed for very particular use-cases (e.g. specific cell line, cell concentration, type of extracellular matrix). Such assays are typically limited in their range of detection or the total number of outputs (e.g. passive tension, developed force, or length-tension relationships) due to design and technical constraints of the platforms utilized. Exemplary arrangements include attaching an engineered tissue construct to a pole and measuring the deflection of the pole after triggering a contraction. Such arrangements can be relatively inaccurate for a number of reasons. For example, as forces increase, the construct becomes dislodged. Furthermore, these arrangements are difficult to modify for different measurement parameters due to fabrication limitations requiring new molds.

Other techniques such as attaching a tissue construct to a rod with two fixed ends have similar issues associated with measurement across a range of force loading and fabrication. Accordingly, there is a need to provide an analysis device which addresses or at least ameliorates some of the above issues and/or provides a potential choice.

### SUMMARY

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

In accordance with a first aspect of the present disclosure, there is provided a sample analysis device for increasing measurement resolution of forces generated by a sample. The sample analysis device may comprise a body stretchable along a central longitudinal axis thereof. The body may comprise at least one attachment region engageable with the sample; a plurality of stretchable portions spaced apart by joining regions; and at least one detectable datum for determining displacement thereof. Each stretchable portion may extend generally longitudinally and include an offset region inclined to the longitudinal axis. The stretchable portions may increase the compliance of the body within a predetermined measurement range.

Optionally, the predetermined measurement range may be modifiable by changing one or more of the parameters selected from the group comprising the number of stretchable portions arranged in a longitudinal direction, the number of stretchable portions transversely extending across the body; and the geometric parameters of the stretchable portions.

Advantageously, the geometric parameters of the stretchable portions may be selected from the group comprising the radius, degree of curvature, length of the offset portion, length of the joining region between adjacent stretchable portions, and width across the offset region.

The geometric parameters of the stretchable portions may be customised for the predetermined measurement range using Finite Element Analysis.

Optionally, the sample may be derived from human or non-human tissue or cells.

Advantageously, the sample may be selected from the group including primary cells, embryonic stem cells, or induced pluripotent stem cells.

The sample may comprise cells and/or tissues selected from the group consisting of skeletal muscle, cardiac muscle, smooth muscle, and non-muscle cells including tissue comprising cells that aggregate or compact over time and are soft and capable of exerting tension, fibroblast tissues, tendon, ligament, and precursor cells or tissues of the liver, stomach, pancreas, gall bladder, kidney, small intestine, colon, urethra, ureter, bladder, prostate, uterus, ovary, eye, skin, brain, tongue, esophagus, or vascular tissue.

The increased measurement resolution of forces may be used for determining parameters selected from the group comprising passive tension/force of the sample; developed force of the sample parameters specifying twitch profile; total force; length-tension relationship including the force with respect to the length of the sample having a Frank Starling mechanism; force-frequency relationship including the beating frequency of the sample; and electrophysiological properties of the sample selected from a group comprising rate of contraction; beat rate variability and indices of arrhythmogenicity.

The predetermined measurement range may be modified by changing one or more of the thickness of the body and the effective elastic properties of the body.

Optionally, the at least one detectable datum may be included between the attachment region and the adjoining stretchable portions.

The at least one detectable datum may comprise one of the following selected from the group comprising a piezo resistive component embedded at a predetermined location in the body; a magnetic material embedded at a predetermined location in the body; a Hall effect sensor; an RFID tag embedded at a predetermined location, an optically contrasting region, a light deflecting region, a light emitting region, a laser deflecting region, a light diffracting region, and a laser diffracting region.

The body may comprise a material that exhibits elastic or hyperelastic behaviour. Advantageously, the body may comprise a polymer or elastomer, optionally, silicone or polydimethylsiloxane.

The sample may exert a contraction force generally along the axis of the body.

The stretchable portions may be arranged in a zig-zag conformation, a serpentine configuration or a rippled configuration.

Optionally, the attachment regions may include geometric features shaped for at least partial encapsulation by overgrowth of sample comprising biological tissue or cells.

The attachment regions may include a plurality of projections therefrom. Advantageously, the attachment regions may include at least one or more holes extending therethrough or formed therein.

Optionally, the device may include at least one member connecting the stretchable portions and extending perpendicular to the central longitudinal axis.

In accordance with a second aspect of the present disclosure, there is provided a biological sample analysis device for measuring forces generated by the biological sample. The biological sample analysis device may comprise a body having a central longitudinal axis and being stretchable along the axis. The body may comprise at least one attachment region engageable with the biological sample; and a plurality of stretchable portions spaced apart by joining regions. Each stretchable portion may extend generally longitudinally and include an offset region inclined to the longitudinal axis. The stretchable portions may increase the displacement of the body for a given force within a predetermined measurement range. The displacement of at least one datum on the body by forces generated from the sample and transmitted across the body may be measurable by a sensor.

The sample may be selected from the group including primary cells, embryonic stem cells, or induced pluripotent stem cells.

Optionally, the sample may comprise cells and/or tissues selected from the group consisting of skeletal muscle, cardiac muscle, smooth muscle, and non-muscle cells including tissue comprising cells that aggregate or compact over time and are soft and capable of exerting tension, fibroblast tissues, tendon, ligament, and precursor cells or tissues of the liver, stomach, pancreas, gall bladder, kidney, small intestine, colon, urethra, ureter, bladder, prostate, uterus, ovary, eye, skin, brain, tongue, esophagus, or vascular tissue.

Optionally, the at least one detectable datum may comprise one of the following selected from the group comprising a piezo resistive component embedded at a predetermined location in the body; a magnetic material embedded at a predetermined location in the body; a Hall effect sensor; an RFID tag embedded at a predetermined location, an optically contrasting region, a light deflecting region, a light emitting region, a laser deflecting region, a light diffracting region, and a laser diffracting region.

The attachment regions may include geometric features shaped for at least partial encapsulation by overgrowth of sample comprising biological tissue or cells.

Optionally, the attachment regions may include a plurality of projections therefrom.

Optionally, the attachment regions may include at least one or more holes extending therethrough or formed therein.

The increased measurement resolution of forces may be for determining parameters selected from the group comprising passive tension/force of the sample; developed force of the sample parameters specifying twitch profile; total force; length-tension relationship including the force with respect to the length of the sample having a Frank Starling mechanism; force-frequency relationship including the beating frequency of the sample; and electrophysiological properties of the sample selected from a group comprising rate of contraction; beat rate variability and indices of arrhythmogenicity.

Optionally, the sample exerts a contraction force generally along the axis of the body.

In accordance with a third aspect of the present disclosure, there is provided a method for measuring properties of a biological sample. The method may comprise culturing cells within an extracellular matrix material in a predefined shape so as to engage with at least one attachment region of a body stretchable along a central longitudinal axis; transmitting force from the cultured cells across the body via a plurality of stretchable portions spaced apart by joining regions; and determining displacement of at least one detectable datum.

Each stretchable portion may extend generally longitudinally and include at least one or more offset regions inclined to the longitudinal axis. The stretchable portions may increase the compliance of the body within a predetermined measurement range.

### BRIEF DESCRIPTION OF THE FIGURES

In order to describe the manner in which the above-recited and other advantages and features of the disclosure can be obtained, a more particular description of the principles briefly described above will be rendered by reference to specific embodiments thereof which are illustrated in the appended figures. Understanding that these figures depict only exemplary embodiments of the disclosure and are not therefore to be considered to be limiting of its scope, the principles herein are described and explained with additional specificity and detail through the use of the accompanying figures.

Preferred embodiments of the present disclosure will be explained in further detail below by way of examples and with reference to the accompanying figures, in which:

FIG. 1A depicts an exemplary tissue construct analysis device according to an embodiment of the present disclosure;

FIG. 1B depicts an enlarged view of the stretchable portions of the tissue construct analysis device of FIG. 1A;

FIGS. 2A-21 depict different exemplary stretchable portions of the present disclosure;

FIG. 3A depicts five different exemplary configurations of the stretchable portions and one control portion;

FIG. 3B illustrates the effects of changes to various geometric parameters such as radius, the degree of curvature and the length of the joining region between adjacent stretchable portions of the configurations of the exemplary stretchable portions of FIG. 3A;

FIG. 3C depicts an enlarged view of the force-displacement relationships in relation to the device of Design #3 to 5 as circled in FIG. 3B;

FIG. 4A depicts an exemplary force-displacement diagram for an analysis device with uniform stretchable portions according to one embodiment of the present disclosure;

FIG. 4B depicts an exemplary force-displacement diagram for an analysis device with non-uniform stretchable portions according to an embodiment of the present disclosure;

FIG. 5A depicts two different exemplary configurations of the analysis devices, with the first device having a single chain stretchable portion and the second having double chain stretchable portions;

FIG. 5B depicts the effects of changes to the number of stretchable portions transversely extending across the polymer body on force-displacement relationship according to the single and double chain stretchable portions in the embodiments depicted in FIG. 5A;

FIG. 5C depicts the effects of changes to the thickness of the body on force-displacement relationship according to the double chain stretchable portions in the embodiment depicted in FIG. 5A;

FIG. 6A depicts the Finite element analysis (FEA) simulations of two different exemplary configurations of the devices in the embodiments depicted in FIG. 5A;

FIG. 6B depicts the force-displacement relationships obtained from the empirical data and the Finite element analysis (FEA) according to the device having double chain stretchable portions in the embodiment depicted in FIG. 6A;

FIG. 7 depicts an exemplary configuration of the analysis device with combined modified parallel and perpendicular components according to an embodiment of the present disclosure;

FIG. 8A depicts an exemplary embodiment of a detectable datum where an opaque dye is used;

FIG. 8B depicts the detectable datum depicted in FIG. 8A where the opaque dye is fluorescent for additional contrast;

FIG. 9A-9E depict exemplary schematic representation of (i) different geometric configurations of the attachment region of the body; and (ii) photographs of corresponding exemplary tissue constructs in the attachment regions;

FIG. 10 depicts displacements of two exemplary tissue construct analysis devices, with the first analysis device having no stretchable portions and the second having a single chain stretchable portions depicted in FIG. 5A;

FIG. 11A depicts the device according to an embodiment of the present disclosure with a PDMS insert;

FIG. 11B depicts the device according to an embodiment of the present disclosure with a layer of medical adhesive backing for selective UV ozone treatment;

FIG. 11C depicts the tissue encapsulation of an exemplary configuration of a single chain stretchable tissue construct device according to the present disclosure seven days after seeding; and

FIG. 11D depicts the tissue encapsulation of an exemplary configuration of a double chain stretchable tissue construct device according to the present disclosure seven days after seeding.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

In a broad aspect of the present disclosure, there is provided a tissue construct analysis device formed from a polymer body having a plurality of stretchable portions which increases displacement of the body for a given force applied to the body within a predetermined measurement range, thus increasing the compliance (inverse stiffness), and thereby the sensitivity of the body.

The movement of the polymer body caused by the tissue construct may be measured by detecting displacement of at least one detectable datum. The tissue construct analysis device is a versatile, easily manufactured and reliable system for monitoring various parameters of the tissue construct under certain physiological conditions.

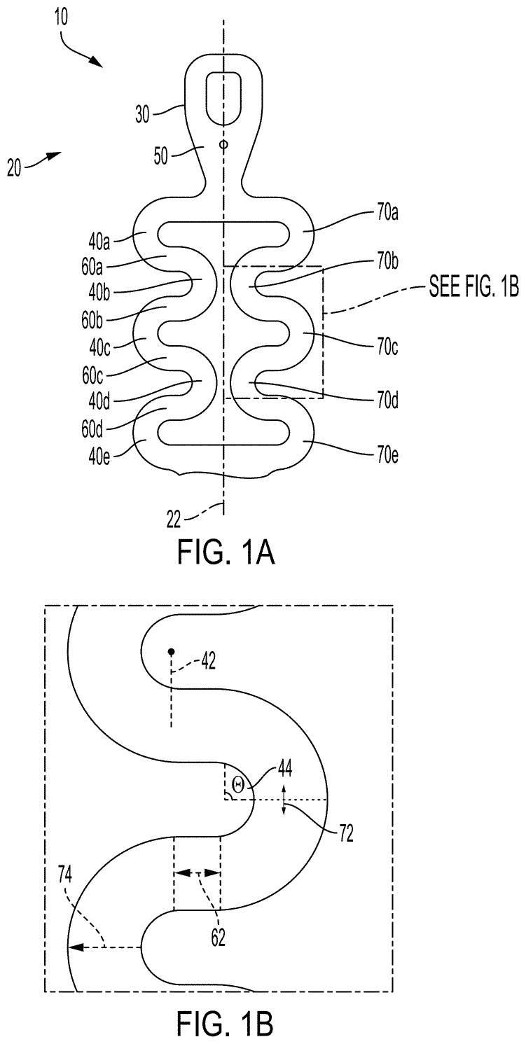

Referring to FIG. 1A, there is depicted an example of an exemplary configuration of a tissue construct analysis device according to the present disclosure. The tissue construct analysis device 10 comprises a body 20 stretchable along a central longitudinal axis 22 of the body. The body 20 comprises at least one attachment region 30, a plurality of stretchable portions 40a, 40b, 40c, 40d, 40e and at least one detectable datum 50 for detecting its displacement. The attachment region 30 is engageable with the tissue, by culturing or similar the tissue as discussed below.

The plurality of stretchable portions 40a, 40b, 40c, 40d, 40e are spaced apart by joining regions 60a, 60b, 60c, 60d. Each of the stretchable portions 40a, 40b, 40c, 40d, 40e extends generally longitudinally and includes an offset region 70a, 70b, 70c, 70d, 70e inclined to the longitudinal axis 22. The stretchable portions 40a, 40b, 40c, 40d, 40e can increase displacement of the body 20 for a given force applied to the body 20 (compliance of the body) within a predetermined measurement range, as is discussed in more detail below.

In one embodiment, the tissue construct analysis device can be coupled to an engineered tissue construct similar to a muscle-tendon unit. For example, the tissue construct may be attached to the device to exert a contraction force generally along the plane of the polymer body of the device. The device is able to measure muscle properties including contractility in an in-plane and auxotonic manner in which the length of the muscle changes and the tension differs due to an increasing load or difference in leverage.

The sensitivity, detection range of forces, and resistance profile (auxotonic relationship) can be easily changed by altering the geometric properties of the stretchable portions of the body, and may be tuned to specific ranges using such alterations. The stretchable portions are capable of amplifying the strain or displacement of the tissue construct analysis device for a given force that the tissue exerts into detectable ranges, provided geometric properties of the device are configured to provide the appropriate sensitivity. By deliberation modification to various geometric properties of the device, it may be configured to have high sensitivity with resolution down to micro-Newtons and a dynamic or changeable range of force detection.

In an example of the present disclosure, the stretchable portions comprise soft elastic polymers to amplify the stretch of the material when low amounts of stress are applied (e.g. the contraction of a tissue construct). This stress is lower by magnitudes of order than that required to break brittle materials.

In order to measure muscle properties consistently, the predetermined measurement range of the device 10 is able to be modified by changing one or more of various geometric parameters including the number of stretchable portions 40 arranged in a longitudinal direction, the number of stretchable portions 40 transversely extending across the polymer body 20, and the geometric parameters of the stretchable portions 40.

In the embodiment depicted in FIG. 1A, the body 20 includes two essentially parallel chains of stretchable portions 40. In the exemplary configuration depicted each chain has five stretchable portions 40a, 40b, 40c, 40d, 40e.

There are five stretchable portions 40a, 40b, 40c, 40d, 40e arranged in a longitudinal direction in FIG. 1A in each of the two adjacent chains of stretchable portions. Thus, in the body shown there are two stretchable portions arranged adjacent to each other. It should be appreciated that the number of stretchable portions 40 arranged in a longitudinal direction and the number of stretchable portions 40 transversely extending across the polymer body 20 can be selected based on practical requirements without departing from the scope of the present disclosure; and according to the desired predetermined measurement range and sensitivity required.

FIG. 1B depicts an enlarged view of the stretchable portions of the tissue construct analysis device of FIG. 1A. The stretchable portions 40 can have geometrical parameters that are generally the same for each; but which can be changed according to the desired measurement range and sensitivity required.

The geometric parameters of the stretchable portions 40 which can be modified include the radius 42, degree of curvature 44, length 72 of the offset portion 70, length 62 of the joining region 60 between adjacent stretchable portions 40, and width 74 across the offset region 70.

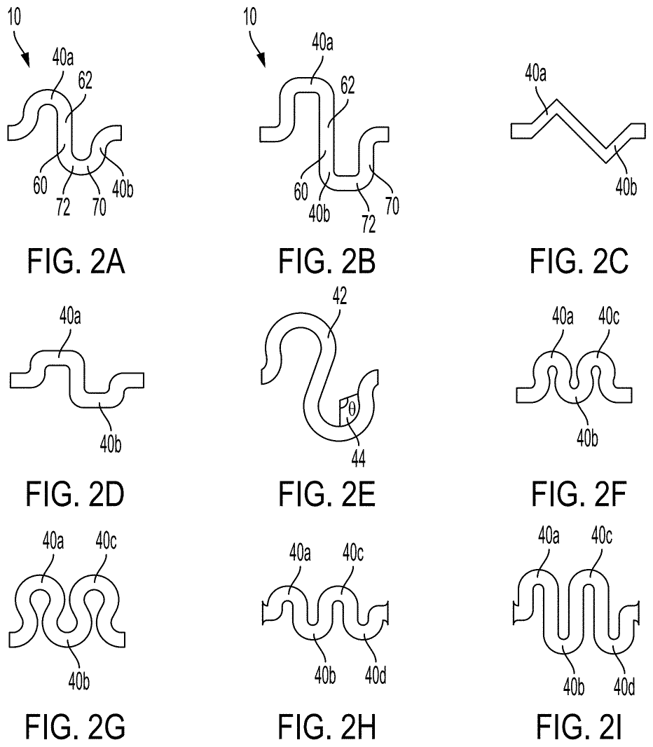

FIGS. 2A-21 depict different exemplary geometric configurations for the stretchable portions of the polymer body of the present disclosure. The stretchable portions 40 of the device 10 can be arranged in a zig-zag conformation, a serpentine configuration, or a rippled configuration as shown in FIG. 2A-21, or other similar configurations having offset regions which are described herein.

Referring to the exemplary geometric configuration as depicted in FIG. 2B, the device 10 shown in FIG. 2B has a greater length 62 of the joining region 60 between adjacent stretchable portions 40a, 40b and a greater length 72 of the offset portion 70 as compared to the device 10 depicted in FIG. 2A.

It should be appreciated that the stretchable portions 40a, 40b of the devices in FIGS. 2C and 2D do not have a circular or radial bends. Instead, the stretchable portion in FIG. 2C has an angular bend.

Comparing the exemplary device configurations of FIGS. 2E and 2A, the device of FIG. 2E has a greater radius 42 and a greater degree of curvature 44 than that of FIG. 2A. The device in FIGS. 2F-2G has three stretchable portions 40a, 40b, 40c while there are four stretchable portions 40a, 40b, 40c, 40d in FIGS. 2H-21. It should be appreciated that the geometric parameters of the stretchable portions of the device can be arranged to provide additional configurations without departing from the scope of the present disclosure.

It should be appreciated that the ratio of displacement to a given force (μm/μN) or sensitivity for the device, along with force sensing range of the device of the present disclosure can be changed by not only altering the device's material/mechanical properties (e.g. Young's modulus) but also by modifying the various geometric parameters of the stretchable portions as previously described.

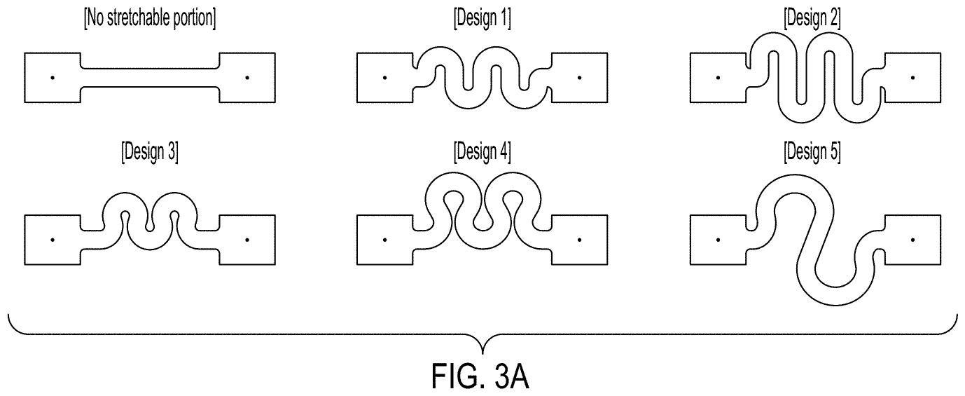

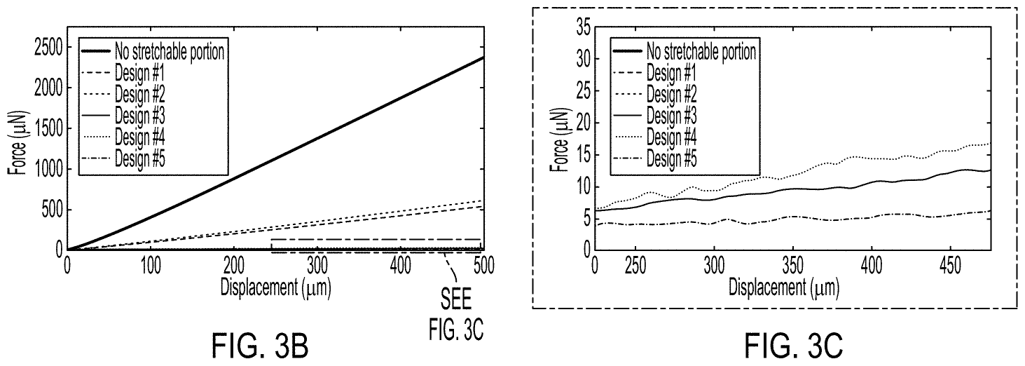

FIG. 3A depicts five different exemplary configurations of the stretchable portions and one control portion of exemplary embodiments of the device.

In a test experiment which was conducted, six devices having different geometric configurations of the devices were fabricated from 100 μm thick silicone sheets as depicted in FIG. 3A. In this experiment, the width 74 across the offset region 70 for all devices was held constant to 750 μm. To empirically measure the force-displacement relationships of each device, an isometric muscle bath system with a force transducer was used (801C, Aurora Scientific). It should be appreciated that no tissues or biological samples were involved in the isometric muscle bath tests system, although the results were used to confirm the FEM results, which are discussed below. The pinholes can be an alternative attachment region.

In this test, each device had pinholes (<500 μm in diameter) on both ends that could be attached or coupled to the lever arms of muscle bath system for empirical measurements of the properties of the device. The distance between the two pinholes was kept constant between all devices at a length of 8.5 mm from center-to-center.

The first one of the six devices having no stretchable portions is set as a reference so as to highlight the amplifying effect of the stretchable portions. For Designs #1 to 5 as depicted in FIG. 3A, Designs #1 and 2 have the same degree of curvature of 90 degrees but Design #2 has a greater length of the joining region between adjacent stretchable portions than that of Design #1; Designs #3 and 4 have a greater degree of curvature but less radius than those of Design #5.

FIG. 3B illustrates the effects of changes to various geometric parameters such as radius, the degree of curvature and the length of the joining region between adjacent stretchable portions of the configurations of the exemplary stretchable portions of FIG. 3A. FIG. 3C depicts an enlarged view of the force-displacement relationships in relation to the device of Design #3 to 5 as circled in FIG. 3B.

Referring to FIGS. 3B and 3C, when the device with no stretchable portions is stretched to 500 μm, its ratio of displacement to a given force was 0.21 μm/μN. Other device Designs #1 to 5 with stretchable portions have a higher ratio of displacement to force ranging from 0.81 to 77.37 μm/μN (3.97- to 366.86-fold higher than that of the no stretchable portion design), which demonstrates the amplifying effect of stretchable portions along with dynamic force sensing range.

Among the device of Designs #1 to 5 with stretchable portions, FIG. 3B shows the device of Designs #1 and 2 have force-displacement relationships that are distinct from those of the other three designs. This could be attributed to that Designs #1 and 2 have a degree of curvature of 90 degree, while the degree of curvature of other three designs are 110 degree or greater.

In comparison between Designs #1 and 2, Design #2 only has a greater length of the joining region between adjacent stretchable portions that does slightly decrease its ratio of displacement to force compared to Design #1 (i.e. 0.90 μm/μN for Design #1 and 0.81 μm/μN for Design #2).

For Designs #3, 4 and 5, it is noted that for all of the devices for these three designs; there is a higher ratio of displacement to force than Designs #1 and 2.

Further, the device of Design #5 has the highest ratio of displacement to force even though it has the smallest degree of curvature among the Designs #3, 4 and 5 and has one less stretchable portion than Designs #3 and 4. This indicates that the increased radius feature of Design #5 (1.75 mm compared to 1 mm and 1.25 mm of Designs #3 and 4 respectively) has a significant impact on the ratio of displacement to force.

The data from this experiment demonstrates that the geometric parameters of the device can be changed so as to detect the displacement within a predetermined measurement range.

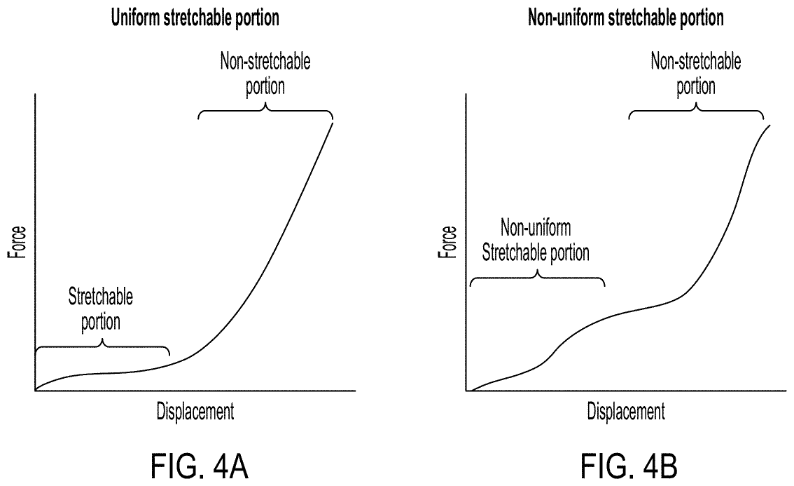

In some embodiments, geometric parameters of only some of the stretchable portions can be changed. Changing a sub-region of some of the stretchable portions can provide the ability to further change the force-displacement relationship if desired. This effectively allow for multiple sensitivity ranges or regions among different lengths. FIGS. 4A and 4B depict exemplary force-displacement relationships for cases with a uniform stretchable portion and a non-uniform stretchable portion, respectively.

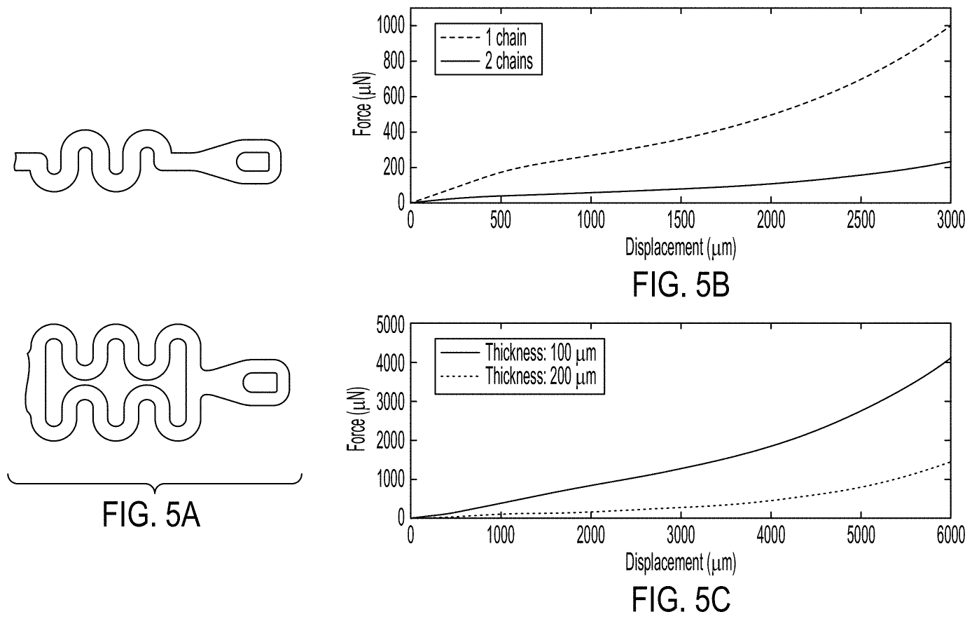

FIG. 5A depicts two different exemplary configurations for tissue analysis devices which are also analysed using the experimental setup used for analysis of the designs of FIG. 3A. For the configurations analysed in experiments on the devices with the configurations depicted in FIG. 5A, the first device included a single chain of stretchable portions while the second device includes two chains of identical arrangements of stretchable portions (i.e. there are two parallel arranged stretchable portions transversely extending across the polymer body).

FIG. 5B depicts the effects of changes to the number of stretchable portions transversely extending across the polymer body on force-displacement relationship according to the single and double chain stretchable portions in the embodiments depicted in FIG. 5A. The device with double chain stretchable portions has a smaller displacement-force ratio, which means that the device with double chain stretchable portions requires more force to displace the same amount of displacement when compared to the device with single chain stretchable portions, as expected.

For the embodiment of the stretchable portions in bodies which were made from silicone, the force-displacement relationship is non-linear as shown in FIG. 5B. As the devices become more stretched, more force is required to displace increments of the same distance, which is very similar to the mechanical properties of native tendon.

Other than changing the geometric parameters of the device, the predetermined measurement range can also be modified by changing the thickness of the body or by changing the effective elastic properties of the body.

The Young's modulus is used to describe the effective elastic properties of a material that exhibits linear elastic behaviour. This effective elastic property is effectively the measurement range of the device. However, it should be appreciated that it is possible that this effective elastic property is nonlinear (hyperelastic materials). In order to change the measurement range, the effective elastic property needs to be changed, assuming geometrical design is held constant. In theory, it is possible to change the material property and not affect the effective elastic property.

FIG. 5C depicts the effects of changes to the thickness of the body on force-displacement relationship according to the double chain stretchable portions of the embodiment of the device depicted in FIG. 5A.

In the experimental results depicted in FIG. 5C, the device with double chain stretchable portions was used to test the effect of thickness of the body on the ratio of displacement to force. It can be seen that as the thickness of the body increases, the cross-sectional area of the body increases, which means more material needs to be displaced and the ratio of displacement to force will decrease. In this embodiment, two thickness conditions (100 and 200 μm) were tested. The experimental data demonstrates that the device with the thickness of 200 μm requires more force to displace a distance of 6 mm (4.12 mN for the thickness of 200 μm compared to 1.42 mN for the thickness of 100 μm). With more material or mass to displace, the device with 200 μm thickness has a lower ratio of displacement to a given force as expected. All the empirical data gathered from the muscle bath system experiments discussed above support the conclusion that the stretchable portions of the device amplify the displacement in response to an acting force for better detection. The response profile (force-displacement relationship) can be easily altered across a dynamic/changeable range by altering the geometric parameters without having to change the material properties. The non-linear response profile can also be changed to mimic that of native human tendon.

While it is demonstrated that the features of the geometrical design can be changed to alter the force-displacement relationship, fabricating and empirically testing on the isometric muscle bath system to obtain a desired force-displacement relationship may be time and resource consuming.

Therefore, other methods for predicting the appropriate geometric parameters for the stretchable portions were investigated. In one embodiment, the geometric parameters of the stretchable portions of the device can be customised for a predetermined measurement range using Finite Element Analysis (FEA). However, it should be appreciated that other methods are possible for determining a predetermined measurement range (a desired force-displacement relationship) in relation to the various geometrical parameters of the device without departing from the scope of the present disclosure.

FIG. 5A depicts the Finite element analysis (FEA) simulations of two different exemplary configurations of the devices in the embodiments depicted in FIG. 4A. In the exemplary experiment of FIG. 5A, FEA simulations of the single chain and double chain stretchable portion devices under static load were performed with both Solidworks and Fusion360 (Dassault Systemes; Autodesk). The simulations assumed a Mooney-Rivlin model of a hyperelastic material. Initial model inputs were 0.6 and 0.6 MPa for the C10 and C01 constants based on a study (LCS Nunes (2011). Mechanical characterization of hyperelastic polydimethylsiloxane by simple shear test. Material Science and Engineering A. 528, 1799-1804). C10 and C01 are material constants for a Mooney-Rivlin model in which the material is assumed to have hyperelastic properties.

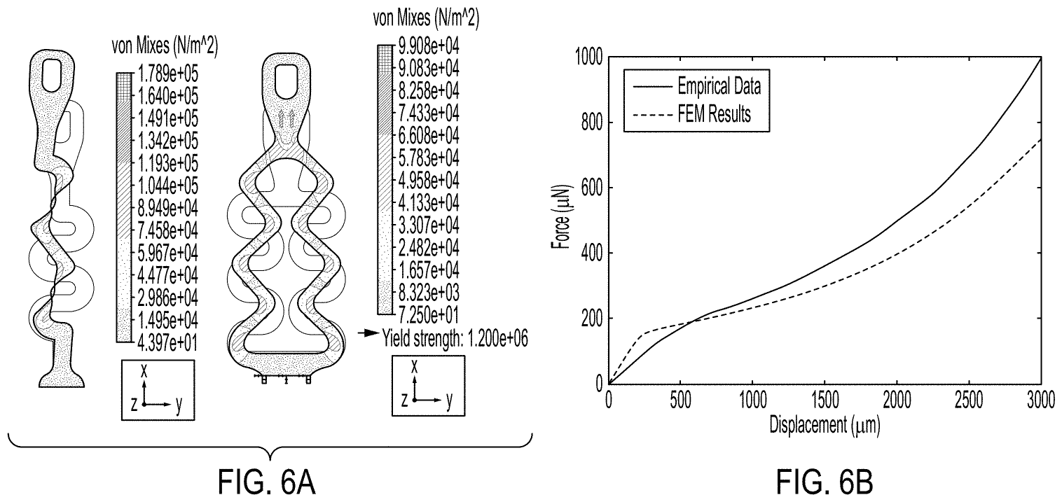

In FIG. 6A, when the devices were simulated to have a load of 500 μN applied in the x-axis direction at the tissue attachment region, the 1-chain design exhibited a translation in the y-axis. For the double chain stretchable portion device, this off y-axis movement was negated and the displacement only occurred along the centerline of the device in the x-axis.

These simulations suggested that the double chain design of the device is more desirable as in this case tracking the displacement of the device is simpler without having to account for off y-axis translation. In addition, the FEA simulations for both designs had out-of-plane motion in the z-axis among the stretchable portion regions. This was visually confirmed during the isometric muscle bath experiments as well.

FIG. 6B depicts the force-displacement relationships obtained from the empirical data and the Finite element analysis (FEA) according to the device having double chain stretchable portions in the embodiment depicted in FIG. 5A.

Subsequently, the double chain stretchable portion device was further simulated to undergo a load of 1000 μN. When compared to the equivalent empirical data, the response profile from the FEA was similar in non-linear behavior and of the same order of magnitude to that of the empirical data as depicted in FIG. 6B. These initial results suggested that FEA could be used to systematically iterate through geometrical designs and accelerate the process to achieve a specific response profile. To improve the results of the FEA, the Mooney-Rivlin constants can be derived from the thin silicone sheets that were used.

While multiple chains as shown in FIGS. 5A and 6A highlight the ability to include multiple geometrical structures in the longitudinal direction parallel to the axis of stretching, it is also possible to add any structure perpendicular (or other oblique angles) to the longitudinal axis of stretching connecting these multiple chains.

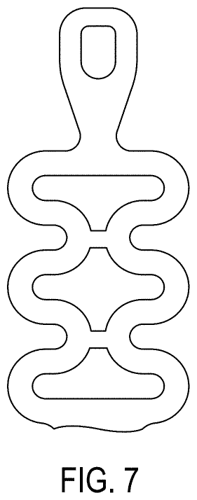

In one embodiment, the device can include at least one members for connecting the stretchable portions and extending perpendicular to the central longitudinal axis. For example, a combination of parallel and perpendicular geometrical parameters result in the exemplary form of a mesh-like structure as shown in FIG. 7. It should be appreciated that other forms would also be possible without departing from the scope of the present disclosure. The at least one detectable datum of the device can be used to quantify properties. In one embodiment, the at least one detectable datum may be located between the attachment region of the tissue construct and the adjoining stretchable portions as depicted in FIG. 1A. However, it should be appreciated that the at least one detectable datum is possible to be located anywhere without departing from the scope of the present disclosure.

In the initial and majority of the device elongation, most of the stretching will occur in the stretchable portions as they can delocalize stress. Thus, the shape of the detectable datum will remain mostly in intact and will experience substantially the same translation as the tissue attachment region, resulting in accuracy of true displacement by the device as a whole. The displacement can then be subsequently converted to force by using the force-displacement relationship established with empirical data from the isometric muscle bath or from the aforementioned FEA data.

In some embodiments, the at least one detectable datum may comprise a piezo resistive component embedded at a predetermined location in the body; a magnetic material embedded at a predetermined location in the body; a Hall effect sensor; an RFID tag embedded at a predetermined location, an optically contrasting region, a light deflecting region, a laser deflecting region, a light diffracting region, and a laser diffracting region. These embodiments are described in detail as below. However, it should be appreciated that the at least one detectable datum can be in any form without departing from the scope of the present disclosure.

In one embodiment, to quantify muscle properties such as passive tension and contractility, an optical approach is utilized where a custom LabView script tracks the detectable datum as depicted in the embodiment of the device of FIG. 1A relative to a stationary background. The detectable datum can be made during the laser cutting portion of the device fabrication in which a distinct pattern is either cut out or etched into the device. The main criteria for the detectable datum is to make it contrasts distinctly enough with the background. To help achieve this criteria, the LabView script can further threshold the acquiring videos to enhance the detection of the detectable datum.

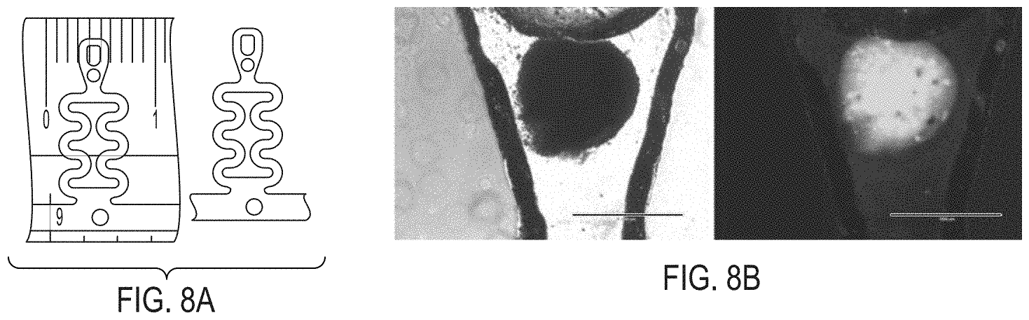

Another strategy is to use an opaque dye (as shown in FIG. 8A). FIG. 8A depicts an exemplary embodiment of a detectable datum where an opaque dye is used; and the opaque dye in FIG. 8B is fluorescent for additional contrast. For example, using the laser mark as a guide for detectable datum location, a 30G needle is dipped into SmoothOn Ignite blue dye and pressed down on the marked point to create a circle that is roughly 1 mm in diameter. Dye is allowed to dry before a thin layer of PDMS is applied to the top using the same needle to seal in the dye. The tissue construct analysis devices are baked to complete encapsulation of the detectable datum such that it is robust and does not rub off during culture. The spot is more easily contrasted from the surrounding transparent device and module in the threshold mode. Due to the fluorescent properties of the dye, additional contrast can be achieved and allows for fluorescent tracking and capabilities (FIG. 8B). Other non-fluorescent silicone based dyes could be used for this detectable datum as well.

An alternative approach in measuring the displacement of the device due to an exerted force (e.g. muscle contraction) is to have a piezo-resistive component embedded in the device. Based on the geometry of the piezo-resistive component relative to the geometry of the device, if a current is applied, the detected resistance should increase as the device is elongated.

Still another approach is to have a magnetic material embedded at the location of the detectable datum. In such an embodiment, by using a Hall effect sensor, displacement can be tracked.

Radio frequency-based technology may also be used in which a tag can be embedded into the region of the detectable datum and with a reader, the displacement can be tracked. Motion tracking devices such as an accelerometer can be similarly embedded. Optionally, other light or laser based technologies can be employed in which a transmitter shines the source onto the device and a receiver is positioned to capture the delay in which the light or laser is either deflected or diffracted based on the motion of the device. Still further, it should be appreciated that a light emitting region, such as fluorescent or other photo-luminescent marker could be utilised as the detectable datum.

In all of these detection modalities, the displacement of at least one or more detectable datum is monitored.

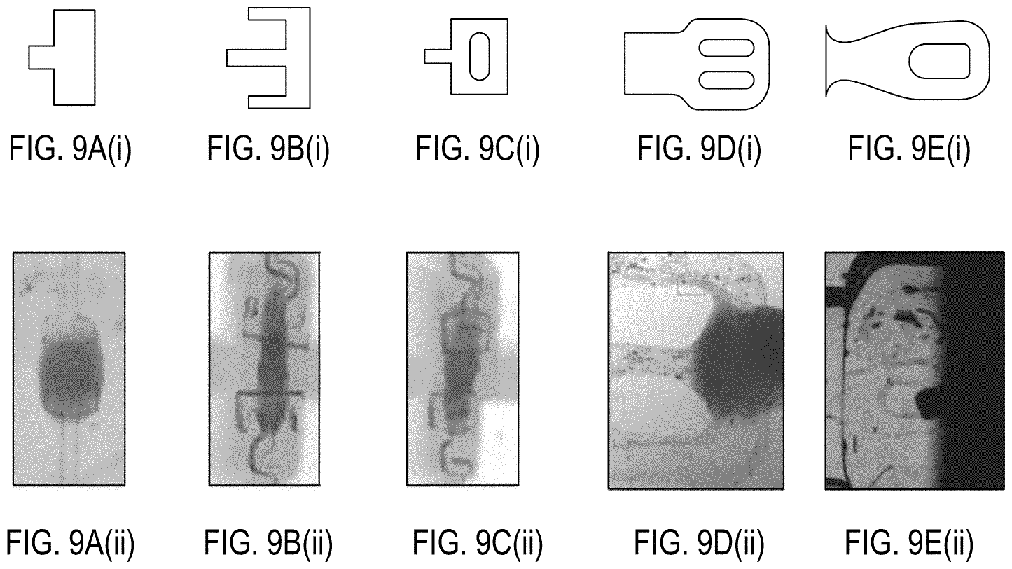

FIG. 9A-9E depict different exemplary geometric configurations for the attachment region of the body to which the muscle tissue attaches. Generally, the attachment regions of the device include geometric features shaped for at least partial encapsulation by overgrowth of the muscle tissue.

For example, as depicted, the attachment regions include a plurality of projections to which the tissue is able to overgrow. FIG. 9A shows that the attachment region (the anchor point) may be a rectangular “bar” which the tissue encapsulates and attaches to. This “bar” design can be modified to have hooks to form the “anchor” shape of FIG. 9B. This anchor shape can improve long term tissue attachment by preventing slippage of tissue off the attachment region.

In another embodiment, the attachment region may also include hole(s) extending through or formed within. As shown in FIG. 9C-9E, the attachment region may also include through-holes which enable the tissue to penetrate through rather than simply wrapping around the attachment point. It should be appreciated that the through-holes of the attachment region may be varied in number and shape (e.g. aspect ratio).

In a further embodiment, the device can have at least one attachment region on each end. In this case, it is possible to have a series or unit in which the device is in between two samples. This could allow for the simultaneous measurement of the sum of the forces generated by both samples.

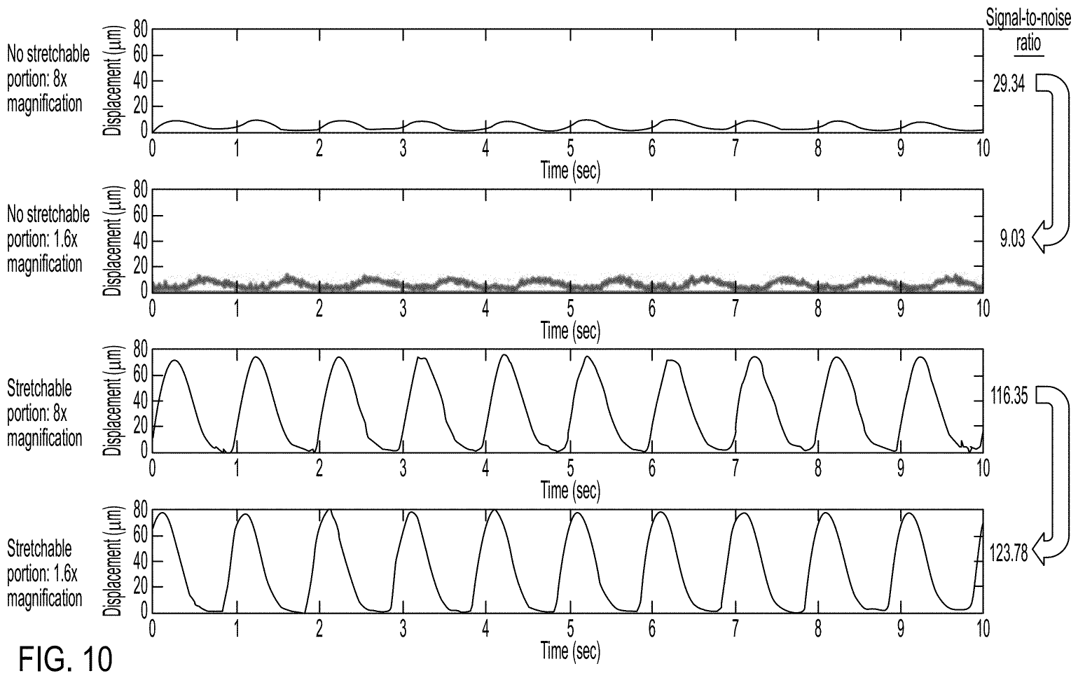

FIG. 10 depicts displacements of two exemplary tissue construct analysis devices, with the first analysis device having no stretchable portions and the second having a single chain stretchable portions depicted in FIG. 5A.

Once the muscle tissues are formed on and attached with the device, the displacement of the device by the contractions of the tissue constructs can be detected.

In one experiment, tissue constructs with the 1-chain stretchable portion design (FIG. 5A) are compared to those with no stretchable portions (i.e. only a tissue attachment point).

As seen in FIG. 10, the detected displacement in the tissue with stretchable portions is noticeably larger than that of the one without stretchable portions, similar to the data obtained using the isometric muscle bath discussed above. This can be seen at both magnifications displayed.

For this analysis, an optical approach was used to track displacement caused by the muscle contraction. The resolution of the device was examined by acquiring images with different magnifications (8× and 1.6×).

For the tissue attached to a device with no stretchable portions, the corresponding signal had a decrease in signal-to-noise ratio from 29.34 to 9.03 when the magnification was decreased from 8× to 1.6×. It should be understood that signal-to-noise ratio is defined as the amplitude of a contractile event relative to the noise of the signal (high frequency components of the signal).

For the tissue attached to a device with stretchable portions, the signal-to-noise ratio remained approximately the same (116.35 to 123.78) when the magnification was decreased.

This suggests that besides amplifying the displacement to a given force, the stretchable portions can provide better resolution and allow for increased flexibility in imaging setup for optical approaches.

In one embodiment, the body can comprise a material that exhibits elastic or hyperelastic behaviour. For example, the body can comprise materials include but not limited to natural or synthetic rubbers (elastomers) and hydrogels or other materials that demonstrate elastic-like properties. Preferably, the body can comprise a polymer or elastomer.

Optionally, the body of the device may comprise silicone. Preferably, the body comprises polydimethylsiloxane. For example, the material used for the above experiments were silicone rubber/elastomer blends. Preferably, the material is bio-inert to minimise the influence on the biological samples (e.g. cells/tissues) being measured, such as the polydimethylsiloxane (silicone-based rubber/elastomer) used to minimize influence on the tissue being measured.

It should be appreciated that the body could be made from any synthetic or natural material or substrate that exhibits elastic or even hyperelastic behaviour with a Young's modulus in the MPa range or lower (for example, in the range of 1 kPa to 100 MPa), and which has minimal plastic deformation. As no material is truly elastic (viscoelastic), it should be appreciated that the substrate may have minimal relaxation time in its stress relaxation properties. Potentially, the material of the body may comprise polyacrylamide hydrogels that are used to create soft elastic surfaces for cell culture.

However, it should be appreciated that the body may comprise other materials without departing from the scope of claims of the present disclosure including material having minimal plastic deformation. It should be appreciated that the materials may include polyacrylamide hydrogels that are used to create soft elastic surfaces for cell culture.

The exemplary devices may be fabricated a number a different ways.

For the test results displayed in FIG. 10, the device was created by creating the geometric configurations in AutoCAD (Autodesk). A 60-Watt CO2 laser cutter (VLS6.60, Universal Laser Systems) was used to cut the devices from thin silicone (polydimethylsiloxane) sheets.

The sheet thickness of 50, 100, 200, 300 μm have been cut utilised; however the fabrication with thicker and thinner sheets could also be performed. The thin silicone sheets used are also rated to have a Young's Modulus of −1.2 MPa. Fabrication with silicone sheets with properties of lower or higher moduli should be possible as well.

The thin silicone sheets are commercially purchased but can be custom mixed or doped for specific material properties and subsequently, spin- or drop-casted to desired thickness. Alternative fabrication strategies could include using a die-cut mechanism, vinyl cutter, waterjet apparatus or injection molding.

A further aspect of the present disclosure also relates to a method for measuring properties of a biological sample.

Generally, the method comprises culturing cells within an extracellular matrix material in a predefined shape so as to engage with at least one attachment region of a body stretchable along a central longitudinal axis; transmitting force from the cultured cells across the body via a plurality of stretchable portions spaced apart by joining regions; each stretchable portion extending generally longitudinally and including at least one or more offset regions inclined to the longitudinal axis.

The stretchable portions increase the compliance of the body within a predetermined measurement range.

The method also comprises determining displacement of at least one detectable datum.

The below describes more particularly a specific aspect of this method, presenting an example of biological data including cardiomyocyte preparation, preparation for tissue formation, preparation of cell solution and seeding.

Cardiomyocyte Preparation

Human embryonic stem cells (hESC), using the hES2 cell line, were differentiated into human ventricular-like cardiomyocytes (hvCMs) based on the embryoid body method.

In brief, cells were maintained on Matrigel coated plates with mTeSR1 at 37° C. with 5% CO2. On the first day of differentiation, cells were digested to form small cell clusters suspended in mTeSR1 with Matrigel, 1 ng/ml bone morphogenetic protein 4 (BMP4) and 10 μM ROCK inhibitor Y-27632 (RI) for 24 hours in an ultra-low attachment plate under hypoxic conditions. Medium was then replaced with StemPro-34 medium with GlutaMAX supplemented with 50 μg/mL ascorbic acid, 10 ng/mL activin A, 10 ng/mL BMP4, and 5 μM RI. After 3 days, cells were cultured in StemPro-34 medium supplemented with 50 μg/mL ascorbic acid and 5 μM IWR-1 for 4 days. Afterwards, cell clusters were maintained in RPMI 1640 supplemented with B27 and 50 μg/mL ascorbic acid in normoxic condition until the day of tissue fabrication. Batches were assessed with flow cytometry on differentiation day 13 or 14 for cardiac troponin T-positive cells, with a quality control criterion of at least 60% cTnT+ cells.

Preparation for Tissue Formation

After fabrication of devices, the devices were washed in a soap water bath with agitation to remove residues from laser cutting. All devices were marked for tracking the displacement as described above and soaked in 70% ethanol. Devices were removed from ethanol and positioned on a 6 mm thick acrylic slab such that the device heads extended just over the edge of the slab. A layer of medical adhesive backing was applied to cover the device bodies and secured with masking tape such that it left the device heads exposed on both sides.

FIG. 9A depicts the device according to an embodiment of the present disclosure with a PDMS insert; FIG. 9B depicts the device according to an embodiment of the present disclosure with a layer of medical adhesive backing for selective UV ozone treatment.

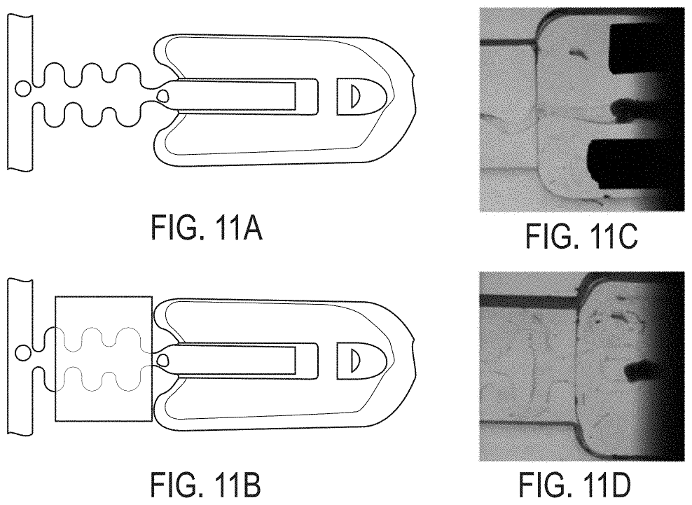

Devices were UV ozone treated for 30 minutes (FIG. 11B). Devices were then fixed by clamping it between two solid layers with the stretchable portions and the tissue attachment region being placed into a well or trough. PDMS inserts were used to limit the volume around the devices such that the tissues were able to encapsulate the device heads (FIG. 11A). Before seeding, 180 μL of 2% BSA in PBS solution was added to each device well, avoiding bubble formation. The module was incubated at 37° C., 5% CO2 for 1 hour then the BSA solution was aspirated. The setup was allowed to air dry prior to seeding.

Preparation of Cell Solution and Seeding

Cardiospheres (hPSC-CM) of directed cardiac differentiation were dissociated on day 15 using 0.025% TE and allowed to reaggregate in suspension in RPMI+B27 supplement (with Ascorbic Acid and ROCK inhibitors) for 72 hours prior to the day of seeding. Each tissue formed around a sensor required 1×106 to 1.3×106 hPSC-CMs. Human foreskin fibroblasts (HFF) were harvested from the culture plate using 0.05% TE. Each tissue formed around a device required 0.1×106 to 0.13×106 (10% of hPSC-CM number).

The cellular mixture was formed by combining the following components: 40% of 5 mg/mi collagen, 1.5% 1M NaOH, 9% 10×MEM, 12.5% 0.2M HEPES, 10% DMEM with Newborn Calf Serum (NCS, 10%) and 6-10% Matrigel, then replenishing by ultrapure water to 100%. This collagen mixture was added to the cell mixture (hPSC-CM+HFF) and the total volume was brought up to 180 μL per tissue using NCS media.

This solution was used for seeding by pipetting 180 μL of cell solution into each sensor section (avoiding bubble formation) that had been defined by the PDMS inserts described above. The module was then placed in the incubator for 1 hour prior to topping up with media by adding 30 mL NCS to the module. Tissues were allowed to compact around devices over the next 2-3 days prior to removing PDMS inserts. Tissues attached to devices were ready for testing 7 days after seeding as shown in FIGS. 11C and 11D. FIG. 11C depicts the tissue encapsulation of an exemplary configuration of a single chain stretchable tissue construct device according to the present disclosure seven days after seeding; FIG. 11D depicts the tissue encapsulation of an exemplary configuration of a double chain stretchable tissue construct device according to the present disclosure seven days after seeding.

The device according to the present disclosure is capable of measuring muscle properties in an auxotonic manner. The stretchable portions of the device can amplify the displacement of the device due to a force exerted by the engineered tissue construct. Due to the ability to easily alter geometrical properties of the device's stretchable portions, the sensitivity, detection range of forces, and resistance profile (auxotonic relationship) can be changed to a certain preference.

In addition to amplifying the displacement to a given force, the stretchable portions can provide better resolution and allow for increased flexibility in imaging setup for optical approaches as mentioned above. The device can be incorporated into custom bioreactors or standard culture ware ranging from 35 mm petri dish enclosures to 96-well plates and higher.

The analysis device also have the below advantages over the prior art. In one embodiment, this device is also used for forming cardiac tissue that have 3D environments and anisotropic formation, similar to that of muscle trabeculae (including cardiac trabeculae).

Another advantage of the device is that, there will be more accurate force estimation/calculation as there is minimal out-of-plane or off-axis movement even when the tissue is lengthened or loaded. The direction of the force exerted by the tissue is primarily isolated to the axis in which the tissue is contracting.

The tissue is always at a set and known Z-height as there is no concern that the tissue is changing Z-height over time or after number of contractions (as seen in some of the pole-based designs). This allows for no requirement of an additional view (cross sectional; Z-X axis) of the tissue to measure tissue height on pole; no calibration of acquisition equipment or apparatuses each time for Z-height before measurements; and ease in auto-focusing and optical visualization for longitudinal tracking of passive tension of tissue as it compacts.

This device also has advantage of easy customization with fast change time. For example, unlike pole-based designs (or anything similar requiring a casting process for fabrication), new master or negative molds do not need to be made. Laser cutting or die cutting from a sheet of material is simple and inexpensive for commercial scale up.

Still another advantage of the device is larger range of customization of the force-distance relationship without need to change material property.

In pole-based prior art, certain geometrical designs, which impact the range of change in the force-distance relationship, are limited by the feasibility in fabricating and casting from the molds. In particular, to increase sensitivity, pole height needs to be increased or diameter of pole needs to be decreased. When pole height is increased too much, it generates high-aspect ratios in features that become very challenging to fabricate or cast (including successful delamination) and mechanically unstable. Similarly, if diameter is decreased too much, the required feature in either the master or negative molds may not be possible (3D printing), difficult (micro-machining with drill), or expensive (photolithography).

The device allows the geometry of the at least one attachment region to be independent of the geometry of the region that displaces or flexes for force measurement.

For example, in the prior pole-based designs, when altering the diameter of the pole (to change the force-distance relationship), the interface between the tissue and poles change, which can affect tissue attachment and formation. For example, if a pole becomes wider, the tissue wrapping around the pole can become too thin and not be able to withstand higher loaded forces. However, with the present device, the changes in geometrical design (the different aspects of the stretchable portions) are independent from the attachment region, which does not need to change.

The present device also allows for ease of fabrication and scalable for high throughput leads to low cost. Specifically, the device can be made from commercially available sheets of silicone (or other elastomer) that are well characterized. Changing the material properties in the prior pole designs may require custom mixtures of multi-component elastomers (e.g., polymer and curing agent). In addition, the increased displacement (μm) to force (μN) ratio (i.e., compliance) of the stretchable allows for equipment that are less expensive and more commercially available (precise equipment that need to detect down to μm can be very expensive and have certain drawbacks such as limited field-of-view or range).

The device can also allow for auxotonic loading of tissues (indicating auxotonic loading lead to stronger force development and improved cardiac tissue structures). The auxotonic relationship (force vs. distance) can be changed or customized to mimic the non-linear relationship between a muscle and tendon.

The device of the present disclosure can be used to detect properties of samples including but not limited to: human/non-human; primary or stem cell-derived (embryonic/induced pluripotent); skeletal muscle, cardiac muscle, smooth muscle, and non-muscle cells including tissue comprising cells that aggregate or compact over time and are soft and capable of exerting tension, fibroblast tissues (tendon, ligament) and precursor cells/tissues of the liver, stomach, pancreas, gall bladder, kidney, small intestine, colon, urethra, ureter, bladder, prostate, uterus, ovary, eye, skin, brain, tongue, esophagus, and vascular.

It should be appreciated that in addition to measuring the displacement and force relationship described, the device of the present disclosure can facilitate measurement of other properties of the sample to which it is attached, including passive tension/force of engineered tissue; developed/active force of engineered tissue (if muscle contracts), for example, any parameters that describe the twitch profile/shape, possibly with respect to time (e.g. max ΔForce/ΔTime of the contraction phase of the twitch); total force of engineered tissue (if muscle contracts); length-tension relationship (the force with respect to the length of the engineered tissue; highlighting Frank Starling mechanism); force-frequency relationship (the force with respect to the beating frequency of the engineered tissue); electrophysiological properties of engineered tissues including conduction properties, rate of contraction, beat rate variability (and related indices of arrhythmogenicity).

Advantageously, the sample analysis device of the present disclosure can be used to measure various parameters for samples that are biological or non-biological (including relatively small forces) generated by samples (e.g. forces in the range of 0.1 μN to 100 N). The ability to customise the design and hence sensitivity of the sample analysis device provides a wide applicability.

Advantageously, the sample analysis device can be used to measure or calibrate the material properties of non-biological samples including for various tissue construct devices of the prior art.

The sample analysis device of the present disclosure may be attached to the poles of other sample analysis devices (e.g. pole-based designs). As the device is pulled along with pole at a set distance, by tracking the deflection of the pole and knowing the compliance of the given sample analysis device, the material properties (e.g. stiffness) of the other sample analysis device can be derived.

It should be appreciated that as the sample analysis device is relatively softer than the metal component of a traditional force transducer, there is less of a mismatch between material properties of the sample and the device, possibly allowing for more accurate measurements with softer materials.

This approach should be a low-cost method compared to using a force transducer, atomic force microscopy, or other force sensing techniques.

It should be appreciated that the device of the present disclosure, by amplifying and increasing the displacement of a detectable datum facilitates the measurement of multiple different properties of a sample, across a diverse range of operational parameters. Fabrication and measurement techniques used for monitoring the device are made relatively easy and cost effective; as the device is easily customised as required without needing precise, expensive monitoring and fabrication techniques.

Accordingly, it is envisaged that the device and the method of the present disclosure can facilitate the following:

(1) in vitro drug development & screening, which comprises a) screening a compound for any cytotoxicity (e.g. cardiotoxicity if tissue is cardiac-based), loss of contractile function, or changes to inherent muscle properties (e.g. stiffness); b) testing any compound for therapeutic effects (e.g. cardioactivity) such as increase in developed force or beating frequency; c) can be examining acute as compared to chronic responses to compound exposure; d) can be examining reversible as compared to irreversible damage (e.g. anthracyclines on cardiac contractile function);

(2) any study effects on tissue formation, compaction, physiological properties (passive & active tension of constructs) due to but not limited to physiological processes and responses including multi-cellular interactions, paracrine signaling, biophysical stimuli, etc.; development and morphogenesis; maturation/conditioning strategies, diseased phenotypes and mechanisms, injury model & corresponding recovery treatments and genetic engineering or therapies; and short- and long-term functional monitoring.

(3) recapitulation of diseased or certain cardiac phenotypes via different regimen of mechanical loading, such as hypertrophy and hyperplasia, cardiomyopathy, arrhythmia; excitation-contraction decoupling and o regenerative and reparative processes.

The above embodiments are described by way of example only. Many variations are possible without departing from the scope of the disclosure as defined in the appended claims.

Although a variety of examples and other information was used to explain aspects within the scope of the appended claims, no limitation of the claims should be implied based on particular features or arrangements in such examples, as one of ordinary skill should be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

1. A sample analysis device for increasing measurement resolution of forces generated by a sample, the sample analysis device comprising a body stretchable along a central longitudinal axis thereof, the body comprising:
at least one attachment region engageable with the sample;
a plurality of stretchable portions spaced apart by joining regions, each stretchable portion extending generally longitudinally and including an offset region inclined to the longitudinal axis, the stretchable portions increasing the compliance of the body within a predetermined measurement range; and
at least one detectable datum for determining displacement thereof.

2. The sample analysis device according to claim 1, wherein the predetermined measurement range is modifiable by changing one or more of the parameters selected from a group comprising the number of stretchable portions arranged in a longitudinal direction, the number of stretchable portions transversely extending across the body, and geometric parameters of the stretchable portions.

3. The sample analysis device according to claim 2, wherein the geometric parameters of the stretchable portions are selected from a group comprising a radius, a degree of curvature, a length of the offset region, a length of the joining region between adjacent stretchable portions, and a width across the offset region.

4. The sample analysis device according to claim 3, wherein the geometric parameters of the stretchable portions are customised for the predetermined measurement range using Finite Element Analysis.

5. The sample analysis device according to claim 1, wherein the sample is derived from human or non-human tissue or cells.

6. The sample analysis device according to claim 5, wherein the sample is selected from a group including primary cells, embryonic stem cells, or induced pluripotent stem cells.

7. The sample analysis device according to claim 6, wherein the sample comprises cells and/or tissues selected from the group consisting of skeletal muscle, cardiac muscle, smooth muscle, and non-muscle cells including tissue comprising cells that aggregate or compact over time and are soft and capable of exerting tension, fibroblast tissues, tendon, ligament, and precursor cells or tissues of the liver, stomach, pancreas, gall bladder, kidney, small intestine, colon, urethra, ureter, bladder, prostate, uterus, ovary, eye, skin, brain, tongue, esophagus, or vascular tissue.

8. The sample analysis device according to claim 5, wherein the increased measurement resolution of forces is for determining parameters selected from the group comprising passive tension/force of the sample, developed force of the sample parameters specifying twitch profile, total force, length-tension relationship including the force with respect to the length of the sample having a Frank Starling mechanism, force-frequency relationship including a beating frequency of the sample, and electrophysiological properties of the sample selected from a group comprising rate of contraction, beat rate variability, and indices of arrhythmogenicity.

9. The sample analysis device according to claim 1, wherein the predetermined measurement range is modified by changing one or more of the thickness of the body and the effective elastic properties of the body.

10. The sample analysis device according to claim 1, wherein the at least one detectable datum is included between the attachment region and the adjoining stretchable portions.

11. The sample analysis device according to claim 1, wherein the at least one detectable detectable datum comprises one of the following selected from a group comprising a piezo resistive component embedded at a predetermined location in the body, a magnetic material embedded at a predetermined location in the body, a Hall effect sensor, an RFID tag embedded at a predetermined location, an optically contrasting region, a light deflecting region, a light emitting region, a laser deflecting region, a light diffracting region, and a laser diffracting region.

12. The sample analysis device according to claim 1, wherein the body comprises a material that exhibits elastic or hyperelastic behaviour.

13. The sample analysis device according to claim 12, wherein the body comprises a polymer or elastomer.

14. The sample analysis device according to claim 1, wherein the body comprises silicone or polydimethylsiloxane.

15. The sample analysis device according to claim 1, wherein the sample exerts a contraction force generally along the axis of the body.

16. The sample analysis device according to claim 1, wherein the stretchable portions are arranged in a zig-zag conformation, a serpentine configuration, or a rippled configuration.

17. The sample analysis device according to claim 1, wherein the attachment regions include geometric features shaped for at least partial encapsulation by overgrowth of sample comprising biological tissue or cells.

18. The sample analysis device according to claim 1, wherein the attachment regions include a plurality of projections therefrom.

19. The sample analysis device according to claim 18, wherein the attachment regions include at least one or more holes extending therethrough or formed therein.

20. The sample analysis device according to claim 1, wherein the device includes at least one member connecting the stretchable portions and extending perpendicular to the central longitudinal axis.

21. A biological sample analysis device for measuring forces generated by a biological sample comprising a body having a central longitudinal axis and being stretchable along an axis, the body comprising:
at least one attachment region engageable with the biological sample; and
a plurality of stretchable portions spaced apart by joining regions, each stretchable portion extending generally longitudinally and including an offset region inclined to the longitudinal axis, the stretchable portions increasing the displacement of the body for a given force within a predetermined measurement range,
wherein the displacement of at least one datum on the body by forces generated from the sample and transmitted across the body is measurable by a sensor.

22. The sample analysis device according to claim 21, wherein the sample is selected from a group including primary cells, embryonic stem cells, or induced pluripotent stem cells.

23. The sample analysis device according to claim 21, wherein the sample comprises cells and/or tissues selected from a group consisting of skeletal muscle, cardiac muscle, smooth muscle, and non-muscle cells including tissue comprising cells that aggregate or compact over time and are soft and capable of exerting tension, fibroblast tissues, tendon, ligament, and precursor cells or tissues of the liver, stomach, pancreas, gall bladder, kidney, small intestine, colon, urethra, ureter, bladder, prostate, uterus, ovary, eye, skin, brain, tongue, esophagus, or vascular tissue.

24. The sample analysis device according to claim 21, wherein the at least one detectable detectable datum comprises one of the following selected from a group comprising a piezo resistive component embedded at a predetermined location in the body, a magnetic material embedded at a predetermined location in the body, a Hall effect sensor, an RFID tag embedded at a predetermined location, an optically contrasting region, a light deflecting region, a light emitting region, a laser deflecting region, a light diffracting region, and a laser diffracting region.

25. The sample analysis device according to claim 21, wherein the attachment regions include geometric features shaped for at least partial encapsulation by overgrowth of a sample comprising biological tissue or cells.

26. The sample analysis device according to claim 21, wherein the attachment regions include a plurality of projections therefrom.

27. The sample analysis device according to claim 21, wherein the attachment regions include at least one or more holes extending therethrough or formed therein.

28. The sample analysis device according to claim 21, wherein the increased measurement resolution of forces is for determining parameters selected from a group comprising passive tension/force of the sample, developed force of the sample parameters specifying twitch profile, total force, a length-tension relationship including the force with respect to the length of the sample having a Frank Starling mechanism, a force-frequency relationship including a beating frequency of the sample, and electrophysiological properties of the sample selected from a group comprising rate of contraction, beat rate variability, and indices of arrhythmogenicity.

29. The sample analysis device according to claim 21, wherein the sample exerts a contraction force generally along the axis of the body.

30. A method for measuring properties of a biological sample, the method comprising:
culturing cells within an extracellular matrix material in a predefined shape so as to engage with at least one attachment region of a body stretchable along a central longitudinal axis;
transmitting force from the cultured cells across the body via a plurality of stretchable portions spaced apart by joining regions, each stretchable portion extending generally longitudinally and including at least one or more offset regions inclined to the longitudinal axis, wherein the stretchable portions increase the compliance of the body within a predetermined measurement range; and
determining displacement of at least one detectable datum.
